(19) United States
(12) Patent Application Publication
(10) Pub. No.: US 2014/0291412 A1
(43) Pub. Date: Oct. 2, 2014

(54) SUNLESS TANNING DEVICE

(71) Applicant: Greg Henson, Lee's Summit, MO (US)

(72) Inventor: Greg Henson, Lee's Summit, MO (US)

(21) Appl. No.: 13/855,442

(22) Filed: Apr. 2, 2013

(51) Int. Cl. A61M 35/00; B05B 15/12

(52) U.S. Cl. CPC A61M 35/00; B05B 15/1222
USPC 239/1; 239/548; 239/302; 239/418; 239/133

(57) ABSTRACT

A spray device and system for delivering a cosmetic composition to a user. The system includes an enclosure booth with an open front, a spray bar with a plurality of spray nozzles configured to deliver a constant volume of cosmetic composition to a user, and a set of heaters designed to deliver warm air to a user during the spray cycle.

### RELATED APPLICATIONS

This application relates to and claims priority to U.S. Provisional Patent Application No. 61/619,283, which was filed Apr. 2, 2012 and is incorporated herein by reference in its entirety.

### BACKGROUND OF THE INVENTION

Sunless tanning, also known as artificial tanning, UV-free tanning, airbrush tanning, or spray tanning, is becoming increasingly popular. Sunless tanning utilizes various chemical compositions that are applied manually or automatically to all or portions of the human body. However, manually applying chemical compositions to the human body frequently results in an uneven coating of the chemical composition. Further, many automatic devices are not comfortable or convenient for a user, and often still fail to apply the chemical composition evenly.

### SUMMARY OF THE INVENTION

Implementations described and claimed herein address the foregoing problems by providing a sunless tanning device that applies an even coat of a chemical composition while being comfortable and convenient for a user.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates a perspective view of an example sunless tanning device.

FIG. 2A illustrates a side view of the example sunless tanning device of FIG. 1.

FIG. 2B illustrates a top view of the example sunless tanning device of FIG. 1.

FIG. 3 illustrates an example application assembly for sunless tanning.

FIG. 4A illustrates a top view of an example bar for applying a chemical composition.

FIG. 4B illustrates a perspective view of the example bar shown in FIG. 4A.

FIG. 4C illustrates a front view of the example bar shown in FIG. 4A.

FIG. 5 illustrates a front view of the example sunless tanning device of FIG. 1.

FIG. 6 illustrates an example air handling assembly for controlling air flow and filtering.

FIG. 7 illustrates a housing for chemical compositions used for sunless tanning.

FIG. 8A illustrates an example non-skid pad.

FIG. 8B illustrates a detailed view of a portion of the example non-skid pad of FIG. 8A.

### DETAILED DESCRIPTION

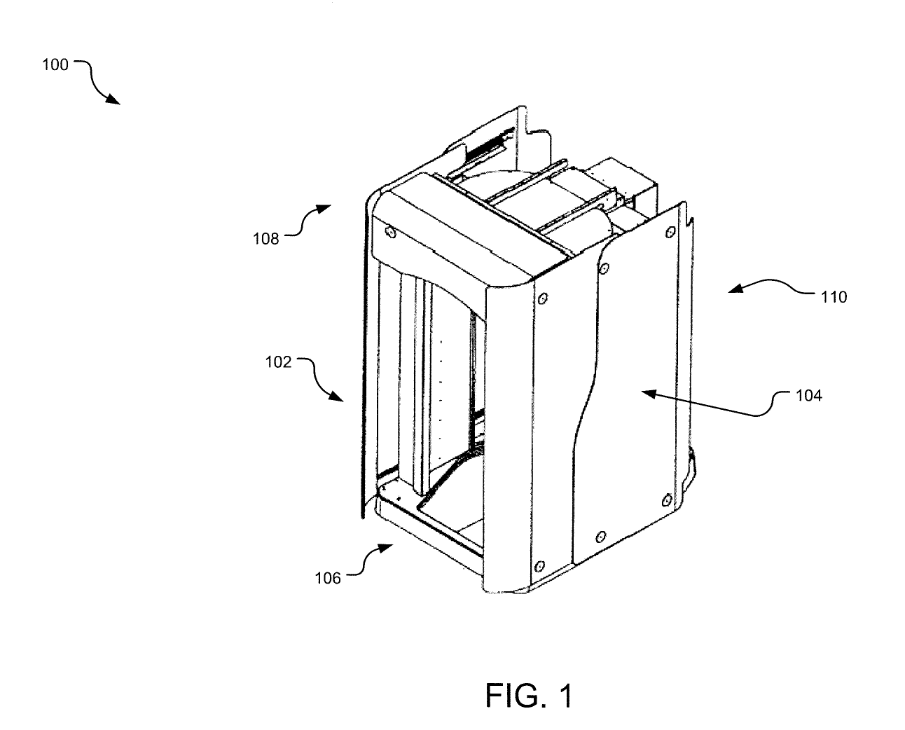

Aspects of the presently disclosed technology involve a sunless tanning device that applies an even coat of a chemical composition such as a cosmetic composition while being comfortable and convenient for a user. As can be understood from a perspective view shown in FIG. 1, an example sunless tanning device or spray system 100 (also referred to as a spray unit) is provided for automatically coating all or selected portions of the human body with a chemical composition, which is generally a fluid. The chemical composition may be a product known in the industry for use on a consumers skin including, for example, but not limited, to a skin darkener, a skin lightener, a skin bleacher, a skin toner, a bronzer, other colorants, a moisturizer, a sunscreen or suntan lotion, a sun-burn or other burn treatment, a wrinkle, fine-line, or other anti-aging treatment, a tanning accelerant, a skin treatment, a cosmetic, an insect repellent, an anti-microbial, an exfoliant, a nutriment or vitamin, a massage aid, a muscle relaxant, a decontamination agent, or some combination of them. However, other chemical compositions that may be applied to part or all of the human body are contemplated. For example, the chemical composition may further include one or more additives, including, without limitation, a preservative, a thickener, a solvent, an emulsifier, a fragrance, a stabilizer, a surfactant, a pH adjuster, an anti-caking agent, or some combination of them.

In one implementation, the sunless tanning device 100 includes an opening 102 through which a user may step to enter the sunless tanning device 100 for use. The opening 102 is defined, in part, by sides 104, a bottom 106, a top 108, and a back 110. The semi-enclosed opening 102 enhances the overall experience of the user. Many automatic devices are completely enclosed, which tends to create a claustrophobic feeling for the user. The opening 102 allows the user to be able to see out of the sunless tanning device 100 during use, thereby reducing or eliminating any claustrophobic feelings. The opening creates the appearance of more space for a user, which increases the overall comfort of use, but does not inhibit the deliver of the chemical composition.

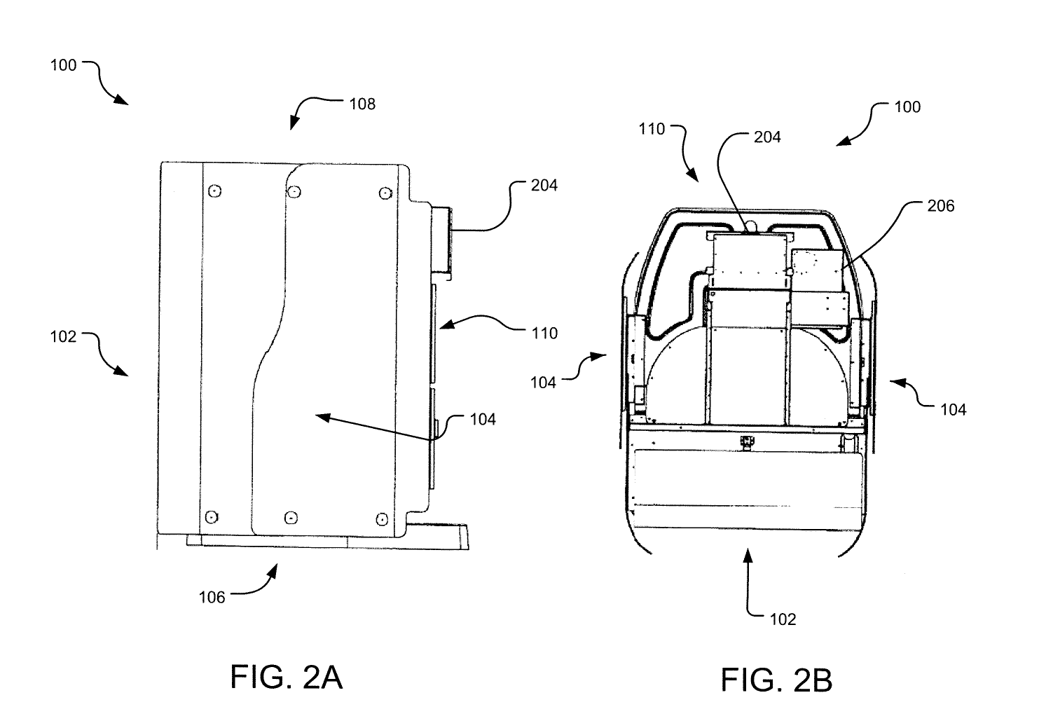

FIGS. 2A and 2B illustrate a side view and a top view, respectively, of the sunless tanning device 100. As shown in FIG. 2A, in one implementation, the side 104 is a substantially smooth surface or plurality of surfaces connecting all or a portion of the base 106 and the top 108. The overall size will depend on the desired configuration of the tanning device and ultimate use. The side 104 may be approximately 96.625 inches tall, the base 106 being approximately 78.5 inches wide, and the top 104 being approximately 58 inches wide. However, other dimensions that may fit within typical salons are contemplated. This allows the positioning of the sunless tanning device 100 in many places and arrangements without having to consider access to the serviceable components or cabinets.

In one implementation, as shown in FIG. 2B, the serviceable components or cabinets, such as cabinet 206, are located at the back 110 of the sunless tanning device 100 near an exhaust unit 204. The components and cabinets are arranged in modules, which allow relatively easy access to the components and cabinets for service. In one implementation, the modules, such as cabinet 206, are removably attached to the sunless tanning device 100, such that the modules may be positioned on one side of the exhaust unit 204 or on the other side of the exhaust unit 204. The ability of the serviceable components or cabinets to be located on either side of the exhaust unit 204 provides flexibility is positioning the sunless tanning device 100 relative to a floor layout or other objects. In another embodiment the modules are integrally attached to the sunless tanning device 100.

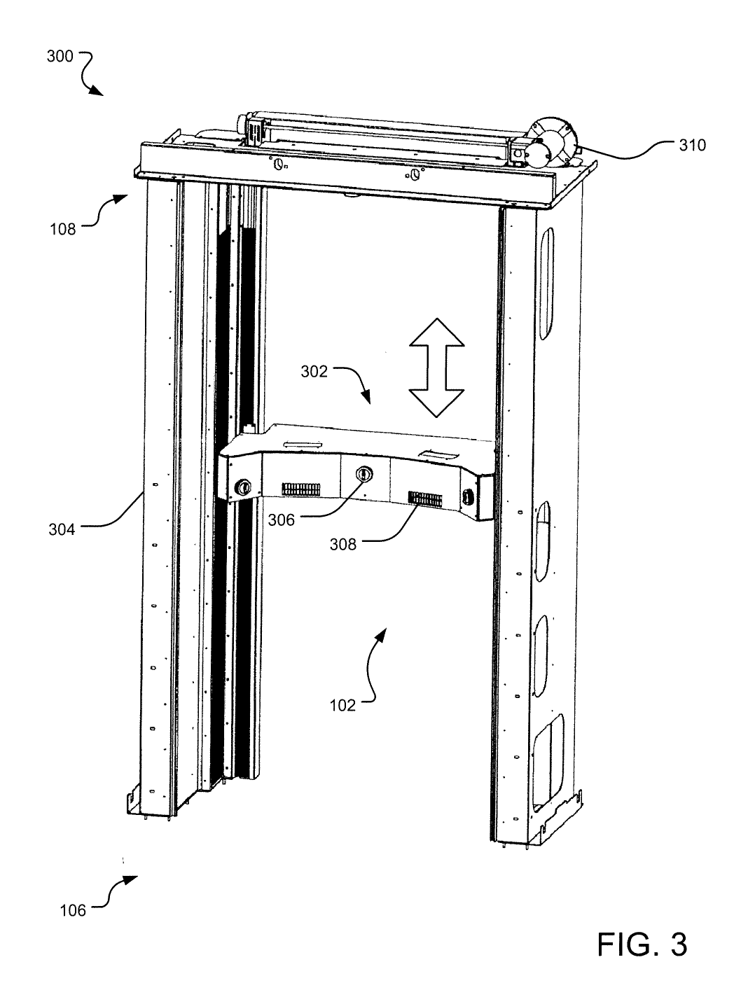

As can be understood from FIG. 3, which shows an example application assembly 300 for sunless tanning, the chemical composition, as described herein, is atomized (e.g., aerosolize, nebulize, or generate mist, fog, or spray) and deposited it on the user. Atomization of the chemical composition generally involves passing the chemical composition through one or more nozzles 306 under pressure. For example, the application assembly 300 may atomize the chemical composition using any method known in the industry, including but not limited to, using air atomization, airless atomization, electrostatic atomization, and ultrasonic atomization. In one implementation, the application assembly 300 atomizes the chemical composition using high volume low pressure (HVLP) forced air delivery. The higher volume of air atomizes the chemical composition and propels it at a low pressure through the nozzles 306, thereby coating it on all or part of the user.

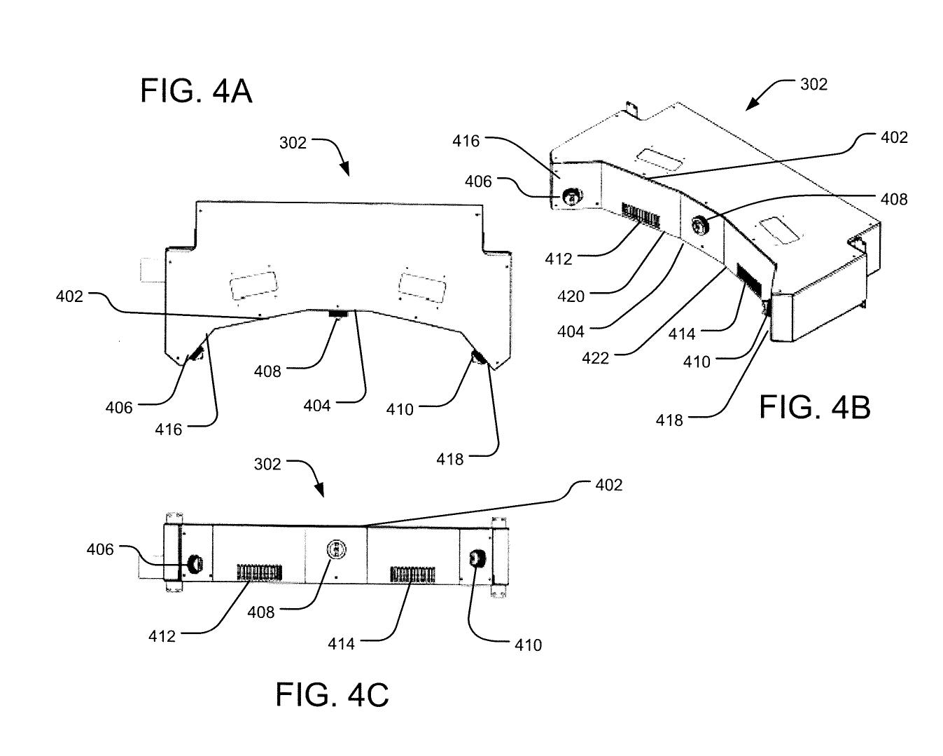

In one implementation, a bar 302 moves vertically along a frame 304. Positioned on the bar is a plurality of the nozzles 306, which are connected to a plurality of pumps (not shown). In one embodiment the bar 302 includes three (3) nozzles 306. In another embodiment the bar 302 includes at least two nozzles 306. In another embodiment the bar 302 includes four (4) nozzles 306. In another embodiment the bar 302 includes five (5) or more nozzles 306. In one implementation, as described with respect to FIGS. 4A-4C, the nozzles 306 are all positioned on different planes of the bar 302. In another embodiment, the nozzles 306 are positioned such that nozzles directly adjacent to each other are located in different planes. Positioning the nozzles 306 on different planes or levels in relationship to each other improves spray patterns by increasing the coverage of each of the nozzles 306 and decreasing interference between the sprays from each of the nozzles 306. Additionally, a coating, such as Dyna Coating, may be applied to the nozzles 306 to reduce the likelihood of clogging and material build up.

As described herein, atomization of the chemical composition is impacted by pressure and volume. Because the chemical composition has to overcome the forces of gravity when traveling from the pumps through the nozzles 306, if the chemical composition is applied at the same pressure as the bar 302 moves from the top 108 to the bottom 106, a greater volume of the chemical composition will be applied on the lower portions of the user's body than the top portions, which results in an uneven application. Accordingly, to ensure an even application, the application assembly 300 delivers a constant volume of the chemical composition as the bar 202 moves from the top 108 to the bottom 106. To overcome the forces of gravity, the chemical composition is delivered using a gradient pressure. Specifically, the application assembly 300 delivers the chemical composition at a relatively higher pressure when the bar 302 is positioned near the top 108 and a relatively lower pressure when the bar 302 is positioned near the bottom 106. By varying the pressure an even constant volume of the composition is delivered through the nozzle.

The volume of a composition delivered through a nozzle will be constant for a cycle of the bar 302 from the top 108 position to the bottom 106 position and return to the top 108. In another embodiment the volume of the chemical composition is variable for a cycle with more or less chemical composition delivered at defined time intervals or sections. The volume of a chemical composition delivered is from 0.1 oz/sec to 1.0 oz/sec. In another embodiment the volume delivered is 0.25 oz/sec to 0.44 oz/sec.

Varying the volume and/or the pressure at which the chemical composition is delivered through the nozzles 306, impacts the spray pattern of the atomized chemical composition. Additionally, altitude and/or viscosity of the composition can impact spray pattern and volume delivered related to pressure. For example, a higher volume of the chemical composition creates a tighter fan shaped spray from the nozzles 306, and a lower volume of the chemical composition creates a wider fan shaped spray from the nozzles 306. Additionally, varying the volume and/or the pressure affects the atomization and application of the chemical composition. For example, the volume and/or pressure may be further modified, without limitation, to reduce overspray, reduce consumption of the chemical composition, increase the proportion of the chemical composition reaching the user, and increase the uniformity of the application of the chemical composition on the user. In one embodiment the pressure is between 0.1 psi and 7 psi. In another embodiment the pressure is between 2.5 psi and 5 psi. Further, in one implementation, the pump pressure is monitored in substantially real time to ensure the pressure of the pumps remains near the target pressure. The sunless tanning device 100 may include, for example, transducers to monitor the pressure of the pumps. If the pressure is outside operational parameters, the pressure of the pumps may be adjusted.

A motor 310 is operably connected to the bar 302 to move the bar 302 vertically along the frame 304. The motor 310 controls the speed at which the bar 302 moves, which affects the volume of chemical composition that is sprayed on the user. Some automatic devices varying the volume or concentration of the chemical composition applied to the user to achieve different levels of tan shades or darkness. As described herein, pressure and volume affect the atomization of the chemical composition, and proper atomization is important to whether the chemical composition is uniformly applied to the user. Further, changing the concentration of the chemical composition creates a risk that the color will not look natural or as intended. Accordingly, rather than varying the pump pressure or volume or changing the concentration of the chemical composition to provide different levels of tan shades, the speed of the motor 310, and thus the bar 302, is varied. Specifically, different levels of tan shades are achieved through regulation of the time it takes for the bar 302 to travel the approximate length of the frame 304 from the top 108 to the bottom 106. For example, a level one (lighter) tan may be correlated to a time of six seconds for the bar 302 to travel; a level two tan may be correlated to a time of seven seconds, and; a level three tan (darker) may be correlated to a time of eight seconds. However, other times and levels are contemplated.

In one implementation, before the bar 302 begins moving, a voice prompt provides the user verbal instructions, including, without limitation, a countdown to advise the user when spray will begin. The voice prompt warns the user to hold their breath and close their eyes. The voice prompt further instructs the user on how to position their body relative to the nozzles 306. Many automatic devices force the user to stand in multiple positions to reach a substantial portion of the user's body, which often results in frustration and mistakes. The presently disclosed technology addresses these problems with the positioning of the nozzles 306, as described with respect to FIGS. 4A-4C. In one implementation, the positioning of the nozzles 306 reduces the number of positions to two positions. Specifically, the user is instructed to stand facing towards the nozzles 306 in a first position and facing away from the nozzles 306 in a second position. Eliminating multiple positions reduces the possibility of user error and increases customer satisfaction. The configuration of the nozzles and overall design of the device 100 allows for a user to only be required to stand in two positions but still allow for uniform coverage of the skin with the chemical composition.

The bar 302 moves in a cycle from a top 108 position to a bottom 106 position. A cycle is considered complete when the bar 302 moves from the top 108 to bottom 106 and then returns to top 108 position. In another embodiment the cycle can include more than a single (multiple passes) from top to bottom to top. The device 100 is designed to deliver a total amount of chemical composition during a cycle. This amount is from 0.1 oz to 5 oz. In another embodiment the amount is 1.5 oz to 3.5 oz.

In addition to reducing possible sources of error based on body positioning, the application assembly 300 includes safety features to detect and respond to possible safety hazards (e.g., a user passing out during use). In one implementation, the sunless tanning device 100 includes one infrared (IR) sensor near the bottom 106 and one IR sensor on an underside of the bar 302 to detect ingress by the user during movement of the bar 302. If all or a portion of the user's body passes under the bar 302 during movement, the IR sensors will detect a potential safety hazard. In response, the motor 310 will stop and move bar 302 back to towards the top 108. Further, the motor 310 may include a load sensor, and if a high current is detected, the motor 310 will stop and move bar 302 back to towards the top 108. In some implementations, after a potential safety hazard is detected, the voice prompt may also notify the user via voice message, visual message, or other message that help is on the way.

Many automatic devices merely spray the chemical composition on the user without any comfort features. For example, many users get cold during application of the chemical composition. Some automatic devices include heaters. However, the heaters often do not adequately direct the heat at the user or the heat is removed too quickly to warm the user during application. Additionally, the chemical composition generally takes a while to dry. Accordingly, the presently disclosed technology provides a comfort forced air drying system.

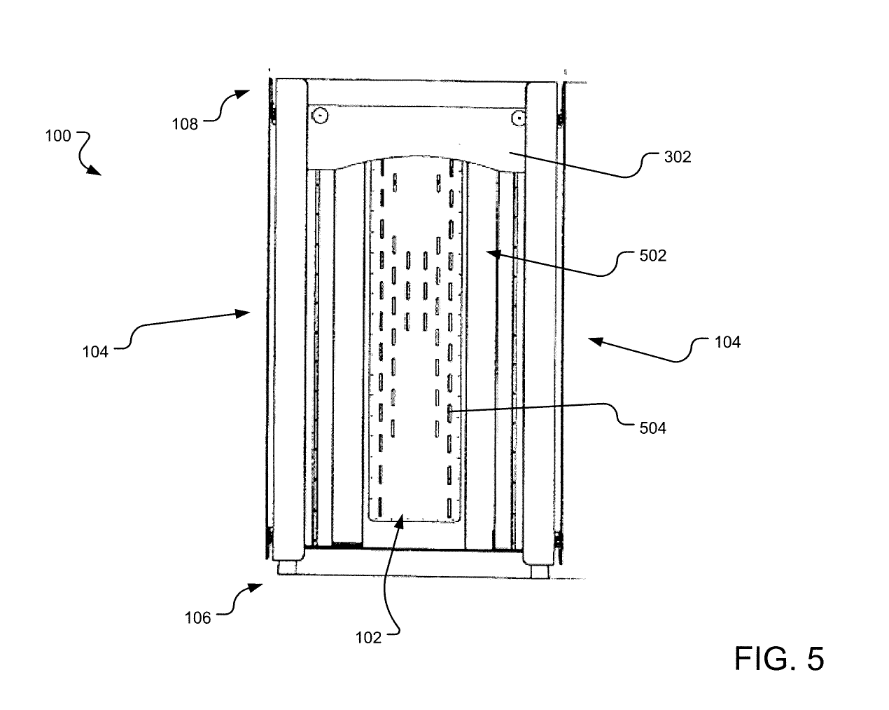

In one implementation, the bar 302 includes one or more heaters 308 to warm and/or dry the user during and/or after application of the chemical composition and to ensure the user remains at a comfortable temperature during user. Prior to a user entering the sunless tanning device 100, the heaters 308 pre-heat the sunless tanning device 100, so the user is comfortable during the entirety of use. While the bar 302 is moving and the nozzles 306 are propelling the chemical composition, the heaters 308 direct forced heated air at the user. In one implementation, the heaters 308 are positioned along the bottom of the bar 302 such that the heated air travels with the atomized chemical composition. For example, the heaters 308 may be positioned in a separate plane from the nozzles 306 or below each of the nozzles 306. The heaters 308 can deliver heated air either prior to or after the chemical composition is delivered. The heaters 308 direct the heated air at a downwards angle relative to the spray patterns of the nozzles 306 to reduce air disruption and vortices impacting the spray patterns. The heaters 308 heat the user's body during delivery of the chemical composition to provide added comfort for the user. Further, the heaters 308 heat the atomized chemical composition after release from the nozzles 306 to dry the chemical composition on the user. While the heaters 308 are providing heated forced air, the exhaust unit 204 is turned off to ensure that the heated air is not removed from the sunless tanning device 100 while the chemical composition is being applied. As described with respect to FIGS. 5-7, unwanted mist resulting from the atomization of the chemical composition is removed from the sunless tanning device 100 prior to initiating the heaters 308.

FIGS. 4A-4C show a top view, a perspective view, and a front view of the bar 302, respectively. The plurality of nozzles 306, as described with respect to FIG. 3, are positioned on a front face 402, which may include one or more surfaces positioned relative to one another at various angles. For example, as shown in FIGS. 4A-4C, the front face 402 may have five angled surfaces. In one implementation, the plurality of nozzles 306 includes nozzles 406, 408, and 410. The nozzles 406, 408, and 410 are placed on the front face 402 at relatively even distances. However, in some implementations, the nozzles 406, 408, and 410 are placed at varying, and sometimes uneven, distances. In one implementation, the nozzle 408 is positioned on a center surface 404 near the center of the front face 402, and the nozzles 406 and 410 are positioned on outer surfaces 416 and 418 of the front face 402 near the edges of the bar 302. The outer surfaces 416 and 418 angle inwards towards the center surface 404, thereby angling the nozzles 406 and 410 inward at the user.

As shown in FIG. 4B, in one implementation, the front face 402 includes a first middle surface 420, disposed between the outer surface 416 and the center surface 404, and a second middles surface 422, disposed between the outer surface 418 and the center surface 404. The heaters 308, described with respect to FIG. 3, include a first heater 412 and a second heater 414 positioned on the middles surfaces 420 and 422, respectively. In additional embodiment more than two heaters are positioned on the spray bar. As described herein, the positioning of the heaters 420 and 422 ensure the heated air travels with the spray from the nozzles 406, 408, and 410 without disrupting the spray pattern.

As can be understood from FIG. 4C, the nozzles 406, 408, and 410 are positioned on different planes of the bar 302, such that the nozzles 406, 408, and 410 are disposed at different vertical positions on the bar 302. For example, in the example implementation shown in FIG. 4C, the nozzle 406 is disposed at a vertical position near the bottom of the bar 302, the nozzle 408 is disposed at a vertical position near the top of the bar 302, and the nozzle 410 is disposed at a vertical position between the vertical positions of the nozzle 406 and the nozzle 408. Positioning the nozzles 406, 408, and 410 on different vertical levels improves spray patterns by increasing the coverage of each of the nozzles 406, 408, and 410 and decreasing interference between the sprays from each of the nozzles 406, 408, and 410. The separation between the vertical positions may range depending on the anticipated position of the user relative to the bar 302. For example, the separation may be approximately 0.10 inches to 2 inches for a user that is anticipated to stand approximately 6 inches to one foot from the bar 302. In additional embodiment the separation is about 0.25 inches to 0.75 inches.

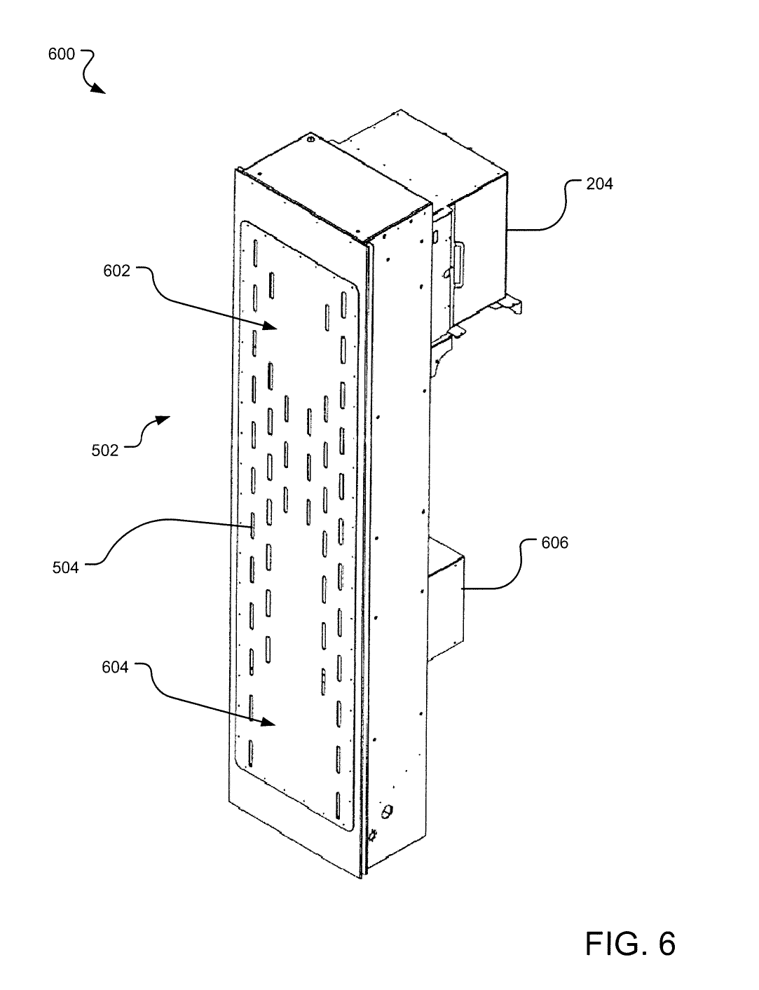

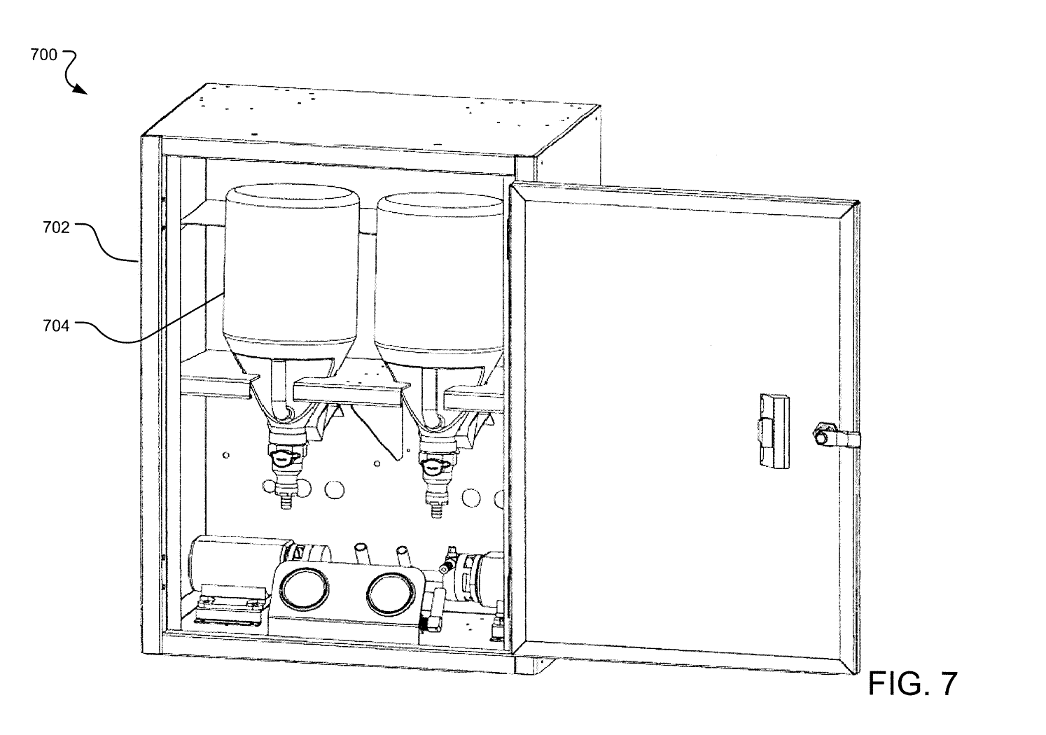

FIG. 5 illustrates a front view of the sunless tanning device 100, which includes a back panel 502 having a plurality of holes 504. As can be understood from FIG. 5, the sides 104, the top 108, the bottom 106, and the back panel 502 contain spray from the bar 302. Containment of the spray reduces waste of the chemical composition, avoids the staining of items outside the sunless tanning device 100, controls air flow, and increases the amount of the chemical composition that is applied to the user. As described in FIGS. 6-7, the air is removed from the sunless tanning device 100 through the holes 504.

As can be understood from FIG. 6, an example air handling assembly 600 controls air flow and filtering. Control of air flow and filtering affects the quality of coating of the chemical composition on the user. Further, the extent of air filtering of the sunless tanning device 100 during a session impacts the quality of the session and user satisfaction. There is a balance between leaving too much spray in the air during a session and drawing the spray out of the sunless tanning device 100 too quickly. If the spray remains in the air too long during a session, the user may be unsatisfied with being surrounded by the spray. Additionally, if the spray is removed through the holes 504 too quickly, the user may not receive an even coating. The air handling assembly 600 includes the exhaust unit 204 and a second exhaust unit 606, which pulls the air through the holes 504. The exhaust units 204 and 606 draw the spray towards the user and any residual spray out of the sunless tanning device 100. Removing residual spray allows the user to breathe easier and be more comfortable without excess spray in his face. Further, removing residual spray decreases maintenance by reducing an accumulation of residue in the sunless tanning device 100.

In addition to the plurality of holes 504, in one implementation, the back panel 502 includes closed section or sections without holes 504, the first closed surface 602 and a second closed surface 604 are configured to improve airflow and delivery of the cosmetic composition to the user. The position of the closed surfaces 602 and 604 on the back panel 502 correlate to body parts of the user, such as the head and legs, respectively. The positioning of the closed surfaces 602 and 604 relative to the holes 504 pull the air from the center of the body and around the upper body parts and the lower body parts to improve the uniformity and quality of the coating of the chemical composition while removing enough residual spray to provide a comfortable user experience.

FIG. 7 illustrates a housing 702 for one or more containers 704 holding the chemical composition. Often, it is challenging for salon attendants to quickly and easily change the containers 704 while tracking the status of the chemical composition. To ensure user satisfaction, the level of fluid in the containers 704 should be monitored to prevent a “dry spray” (i.e., a spray where little to no volume of the chemical composition is propelled from the nozzles 306 onto a customer). In one implementation, the containers 704 each include an associated sensor to detect when the chemical composition in each of the containers 704 is low. In some implementations, the sensor sends a signal to the salon attendant to warn the salon that the chemical composition is low and should be changed soon.

For example, one or more capacitors may be positioned on, in, or near the containers 704 corresponding to different capacity gradients of the containers 704 a range of fluid levels to determine whether the containers 704 are full or close to low. In another implementation, a session count is used to monitor the fluid level of the containers 704, for example, based on the number of sessions since the containers 704 were last replaced. Specifically, the sunless tanning device 100 correlates the number of sessions between when each of the containers 704 is placed in the sunless tanning device 100 and when each of the containers 704 is replaced to predict when the fluid in the container 704 will be low. In still another implementation, sensors that are in direct communication with the fluid in the containers 304 are used to measure fluid levels. Other fluid level sensors are also contemplated. Measuring the fluid levels will also correlate to the total number of sessions administered to a user.

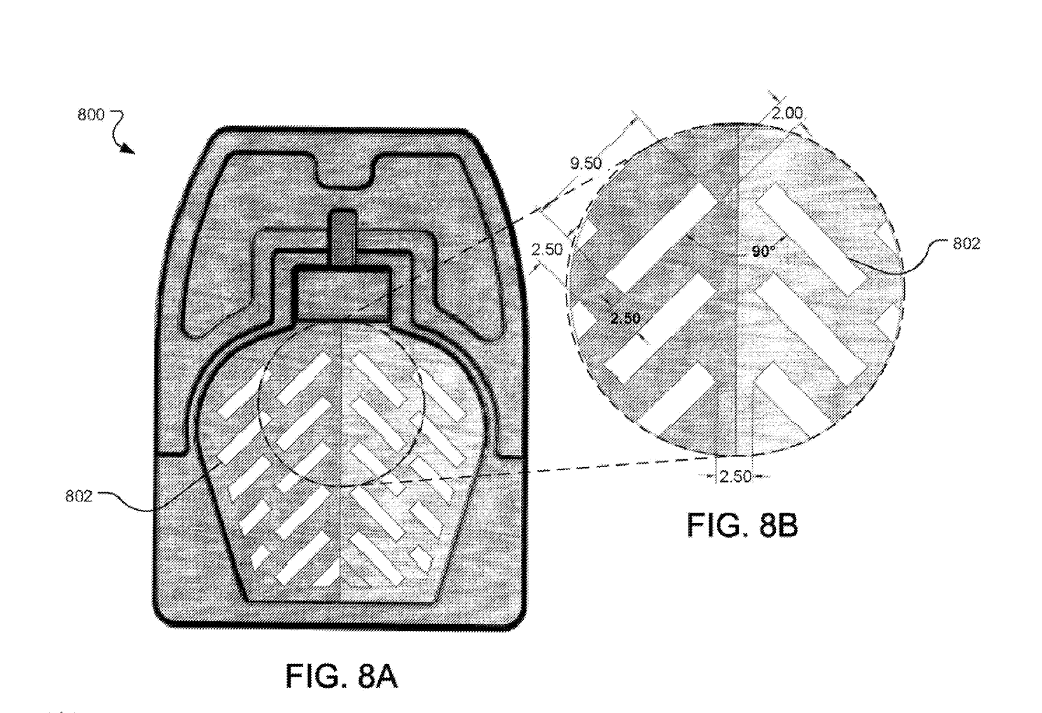

FIG. 8A illustrates an example non-skid pad 800, which includes perforations 802. In one implementation, the perforations 802 are arranged on the non-skid pad 800 in a honeycomb design. However, other arrangements of the perforations 802 are contemplated. The honeycomb structure of the non-skid pad 800 provides reinforcement under the sunless tanning device 100 creating a durable, rigid feel. Further, the honeycomb structure of the non-skid pad 800 catches excess fluid to prevent the fluid from spilling out of the sunless tanning device 100. As shown in FIG. 8B, each of the perforations 802 may be approximately 9.5 inches long and approximately 2 inches wide. Further, the perforations 802 may be placed approximately 2.5 inches away from each other at an approximate 90 degree angle. However, other dimensions and separation distances and angles are contemplated.

The implementations described herein may be implemented as logical steps in one or more computer systems. The logical operations of the presently disclosed technology are implemented (1) as a sequence of processor-implemented steps executing in one or more computer systems and (2) as interconnected machine or circuit modules within one or more computer systems. The implementation is a matter of choice, dependent on the performance requirements of the computer system implementing the presently disclosed technology. Accordingly, the logical operations making up the implementations of the presently disclosed technology described herein are referred to variously as operations, steps, objects, or modules. Furthermore, it should be understood that logical operations may be performed in any order, unless explicitly claimed otherwise or a specific order is inherently necessitated by the claim language. Furthermore, one of more portions of the various processes disclosed above may be implemented by software, hardware, firmware or combination thereof.

The above specification, examples, and data provide a complete description of the structure and use of example implementations of the invention. Various modifications and additions can be made to the exemplary implementations discussed without departing from the spirit and scope of the presently disclosed technology. For example, while the implementations described above refer to particular features, the scope of this disclosure also includes implementations having different combinations of features and implementations that do not include all of the described features. Accordingly, the scope of the presently disclosed technology is intended to embrace all such alternatives, modifications, and variations together with all equivalents thereof.

What is claimed is:

1. A spray system for dispensing a cosmetic composition for skin treatment, the spray system comprising:
a. an enclosure booth, wherein the enclosure booth includes a rear panel, two opposing side panels, a base member, and an open front;
b. a spray bar, wherein the spray bar is designed to communicate with a frame and a track configured to run from a top position to a bottom position on the track; and,
c. a plurality of spray nozzles on the spray bar, wherein the spray nozzles are on separate planes of the spray bar to minimize disruption of the spray pattern of the cosmetic composition.

2. The spray system of claim 1, wherein the rear panel includes a plurality of open holes to allow for the air to be pulled through the holes of the rear panel and exit via the exhaust system.

3. The spray system of claim 1, wherein the rear panel includes a first closed section that does not include any holes and a section closed section also with no open holes, such sections do not include any openings, the first closed section is at the top of the panel and correlates to the head region of a user and the second closed section is at the bottom of the rear panel and correlates to the legs of the user, the configuration of the open front and open holes and closed sections of the rear panel create an air flow path that directs the cosmetic composition across the body surface of the user and exits through the spray system exhaust.

4. The spray system of claim 1, further includes a cabinet configured for attachment to the outside of the spray system enclosure booth, the cabinet further includes housing for holding one or more containers, wherein the container includes the cosmetic composition.

5. The spray system of claim 4, further includes sensors for use with the containers to aide in monitoring the amount of cosmetic composition in the container.

6. The spray system of claim 4, wherein the sensors relay information to a central computer process for remotely monitoring.

7. The spray system of claim 1, further including at least one heater unit on the spray bar.

8. The spray system of claim 7, wherein the heater unit is configured to deliver heated air to the part of the user receiving the cosmetic composition wherein the user receives the heated air before, after, or during the delivery of the cosmetic composition.

9. The spray system of claim 1, further includes a voice prompt device to provide verbal instructions to the user before, after, or during the delivery of the cosmetic composition.

10. The spray system of claim 1, further includes at least one pump for the delivery of the cosmetic composition from the container to the nozzle and onto the user, further at least one pressure or volume monitor is included for monitoring the cosmetic composition in the system, wherein the cosmetic composition is delivered from the nozzle at a volume that remains constant through the cycle of the delivery.

11. The spray system of claim 10, wherein the pressure of the cosmetic composition adjusted throughout the cycle of the cosmetic composition delivery to ensure that a constant delivery of volume of the cosmetic composition remains throughout the cycle delivery.

12. The spray system of claim 9, wherein the volume delivered for the cycle is about 0.1 oz/sec to about 0.8 oz/sec.

13. The spray system of claim 1, further including safety sensors within the enclosure booth.

14. The spray system of claim 13, wherein the safety sensors are IR sensors and are designed to alert the user and return the spray bar to the top position.

15. A spray bar for the delivery of a cosmetic composition to the skin of a user, the spray bar comprising:
a. three spray nozzles, wherein the spray nozzles are on separate planes of the spray bar to minimize disruption of the spray pattern of the cosmetic composition;
b. two heater units, wherein the heater units are configured for placement intermediate the spray nozzles on the spray bar; and,
c. a communication mechanism to allow for the spray bar to communicate with a track of a spray system.

16. The spray bar of claim 15, wherein the nozzles placement on separate planes is from 0.1 inches to 5 inches.

17. The spray bar of claim 15, further including at least one safety sensor.

18. A method for delivering an uniform amount of a cosmetic composition to the skin of a user; comprising:
a. having a user enter an enclosure booth of the spray unit;
b. engaging the spray unit to begin a spray cycle;
c. the user follows the prompts of the spray unit, including facing forward as prompted to allow for the spray cycle to begin;
d. spraying an amount of cosmetic composition from at least two nozzles of a spray system wherein the nozzles are positioned on a spray bar and are placed on separate planes in relationship to each other, wherein the amount of cosmetic composition administered from the at least one nozzle is a constant volume for a spray bar cycle, wherein the spray bar cycle movement of the spray bar is from a top position to a bottom position and return to the top position;
e. the user then turns to face the rear panel and the spray unit delivers an amount of cosmetic composition from at least two nozzles of the spray system to the user through the completion of another spray bar cycle; and,
f. blowing an amount of forced air on the user either prior to, after, or during the delivery of the cosmetic composition.

19. The method of claim 18, wherein after the delivery of the cosmetic composition to the users skin information is delivered to a central computer process for remotely monitoring the spray cycle.
